(19) United States
(12) Patent Application Publication
(10) Pub. No.: US 2021/0095212 A1
(43) Pub. Date: Apr. 1, 2021

(54) CONVERSION OF ETHANE IN SHALE GAS TO VALUABLE CHEMICALS

(71) Applicant: SABIC Global Technologies B.V., Bergen op Zoom (NL)

(72) Inventors: Eswara Rao MUPPARAJU, Bangalore (IN); Sreejit NAIR, Bangalore (IN)

(21) Appl. No.: 16/970,030

(22) PCT Filed: Apr. 4, 2019
(86) PCT No.: PCT/IB2019/052772

§ 371 Date: Aug. 14, 2020

Related U.S. Application Data
(60) Provisional application No. 62/653,453, filed on Apr. 5, 2018.

(51) Int. Cl. C10G 45/68; C07C 2/76; C07C 7/11; B01D 3/38

(52) U.S. Cl. CPC C10G 45/68; C07C 2/76; C07C 7/11; C07C 2529/48; C10G 2300/1025; C10G 2400/30; B01D 3/38

(57) ABSTRACT

A process for producing valuable aromatic hydrocarbons from a crude or semi-crude shale gas stream. A crude or semi-crude shale gas stream including methane is introduced into a reactor that converts at least a portion of the ethane component into aromatic hydrocarbons. Unreacted methane, other hydrocarbons, and hydrogen may then be easily separated from the aromatic hydrocarbons. Because methane is not separated from the shale gas stream, the expensive and resource-consuming shale gas C1/C2+ separation step is avoided.

### CROSS-REFERENCE TO RELATED APPLICATIONS

This application claims the benefit of priority to U.S. Provisional Application No. 62/653,453, filed Apr. 5, 2018, the entire contents of which are incorporated herein by reference in their entirety.

### FIELD OF THE INVENTION

The invention generally concerns methods of producing aromatic hydrocarbons. In particular, the invention concerns the production of benzene, toluene, and xylene aromatic hydrocarbons from a shale gas stream.

### DESCRIPTION OF RELATED ART

Shale is a fine-grained sedimentary rock that forms from the compaction of silt and clay-size mineral particles. Some shales contain organic material such as natural gas and oil. Black organic shales obtain their black color from tiny particles of organic matter that were deposited with the mud from which the shale formed. As the mud was buried and warmed within the earth, some of the organic material was transformed into oil and natural gas. These shales are the source rock for many of the world's large oil and natural gas deposits.

Although conventional drilling can extract large amounts of oil and natural gas from the reservoir rock, much of it remains trapped within shale. This oil and gas is very difficult to remove because it is trapped within tiny pore spaces or adsorbed onto clay mineral particles that make up the shale. In the late 1990s, natural gas drilling companies developed new hydraulic fracturing methods for liberating oil and natural gas trapped within the tiny pore spaces of shale. This discovery was significant because it unlocked some of the largest shale gas deposits in the world.

Although methane is the primary component of all shale gas, the fraction of other constituents, e.g., ethane, propane, nitrogen, water, and CO₂ varies based upon the gas deposit's geographical location. In order to convert shale gas non-methane hydrocarbons into more valuable aromatic compounds, methane is first separated from C2+ hydrocarbons using an expensive cryogenic distillation separation process. Alternatives to cryogenic distillation such as the use of molecular sieves exist, however, any C1/C2+ separation process adds additional expenses to the shale gas reforming process. Thus, there remains a need for improved processes for using shale gas.

### SUMMARY OF THE INVENTION

A discovery has been made that provides a solution to one or more of the problems discussed above. The solution provides a method that circumvents a shale gas C1/C2+ separation step. The method employs a C1/C2+ stream as an input for a reformer or aromatization unit. The reformer or aromatization unit converts a portion of the C1/C2+ stream to heavier aromatic compounds, including benzene, toluene, and xylenes (BTX). These aromatic compounds have a higher average molecular weight than the remainder of the reformer or aromatization unit output stream, and are thus easily separated. Each degree of unsaturation, e.g. ring-formation or double bond formation, introduced produces a molecule of hydrogen. Each combination of hydrocarbon molecules, e.g., 2 H₃CCH₃→H₃C(CH₂)₂CH₃+H₂ also produces a molecule of hydrogen.

The method describe herein eliminates the energy-intensive C1/C2+ separation step, employs a semi-crude C1/C2+ stream as an input for a reformer or aromatization unit, provides a value-added BTX output stream, and facilitates separation of methane components from non-methane components. In some aspects, the creation of hydrogen increases the lower heating value of the non-BTX output stream.

In some aspects, a process for the production of BTX aromatic hydrocarbons from shale gas is provided. The shale gas comprises at least methane and ethane, and may comprise C2+ straight or branched, aliphatic or unsaturated hydrocarbons, or combinations thereof, as well as water, hydrocarbon hydrates, carbon monoxide, carbon dioxide, nitrogen, and other gaseous components. The process comprises the steps of reacting shale gas under conditions sufficient to produce a first stream comprising BTX aromatic hydrocarbons, methane, and hydrogen, and separating the first stream comprising second and third streams.

The second stream comprises methane and hydrogen and the third stream comprises BTX aromatic hydrocarbons. In some aspects, water and hydrates are removed from the shale gas prior to reacting the shale gas. In some embodiments, the shale gas is reacted in a catalytic aromatization unit. In some embodiments, conditions sufficient to produce BTX aromatic hydrocarbons comprise heating the shale gas to a temperature ranging from about 400° C. to about 700° C., preferably from about 450° C. to about 600° C. The reaction pressure may range from 1 bar to 10 bar, preferably from 1 bar to 5 bar. In some aspects, the methane in the shale gas remains essentially unreacted.

In further aspects, a process for the production of BTX aromatic hydrocarbons from shale gas comprises the steps of reacting shale gas comprising methane, ethane, and C2+ hydrocarbons under conditions sufficient to produce a first stream comprising BTX aromatic hydrocarbons, methane, hydrogen, and unreacted ethane and C2+ hydrocarbons, contacting the first stream with a solvent to produce a second stream comprising methane, hydrogen, and unreacted ethane and C2+ hydrocarbons, and a third stream comprising the solvent and the BTX aromatic hydrocarbons, and removing the solvent from the third stream. In some aspects, water and hydrates are removed from the shale gas prior to reacting the shale gas. In some embodiments, the shale gas is reacted in a catalytic aromatization unit. In some embodiments, conditions sufficient to produce BTX aromatic hydrocarbons comprise heating the shale gas to a temperature ranging from about 400° C. to about 700° C., preferably from about 450° C. to about 600° C. The reaction pressure may range from 1 bar to 10 bar, preferably from 1 bar to 5 bar. In some embodiments, the solvent is an aromatic selective solvent, preferably selected from the group consisting of mono-ethylene glycol, di-ethylene glycol, tri-ethylene glycol, tetra-ethylene glycol, tetrahydrothiophene dioxide, N-methylpyrrolidone, dimethylsulfoxide, propylene carbonate, phenol, cresol, N-formylmorpholine, monomethylformamide, N-methyl-ε-caprolactam, water, and combinations thereof. In some embodiments, the methane in the shale gas remains essentially unreacted.

In additional embodiments, a process for the production of BTX aromatic hydrocarbons from shale gas comprises the steps of reacting shale gas comprising methane, ethane, and C2+ hydrocarbons under conditions sufficient to produce a first stream comprising BTX aromatic hydrocarbons, methane, hydrogen, and unreacted ethane and C2+ hydrocarbons, contacting the first stream with a solvent to produce a second stream comprising methane, hydrogen, and unreacted ethane and C2+ hydrocarbons, and a third stream comprising the solvent and the BTX aromatic hydrocarbons, distilling the third stream to produce a product stream comprising the BTX aromatic hydrocarbons and a fourth stream comprising the solvent, and recycling the solvent to the step of contacting the first stream with a solvent. In some aspects, water and hydrates are removed from the shale gas prior to reacting the shale gas. In some embodiments, the shale gas is reacted in a catalytic aromatization unit. In some embodiments, the methane in the shale gas remains essentially unreacted. In some embodiments, conditions sufficient to produce BTX aromatic hydrocarbons comprise heating the shale gas to a temperature ranging from about 400° C. to about 700° C., preferably from about 450° C. to about 600° C. The reaction pressure may range from 1 bar to 10 bar, preferably from 1 bar to 5 bar. In some embodiments, the solvent is an aromatic selective solvent, preferably selected from the group consisting of mono-ethylene glycol, di-ethylene glycol, tri-ethylene glycol, tetra-ethylene glycol, tetrahydrothiophene dioxide, N-methylpyrrolidone, dimethylsulfoxide, propylene carbonate, phenol, cresol, N-formylmorpholine, monomethylformamide, N-methyl-ε-caprolactam, water, and combinations thereof. In some aspects, the BTX aromatic hydrocarbons are further processed to recover benzene. The BTX aromatic hydrocarbons may be distilled to individually collect benzene, toluene, xylenes, and/or other C8+ aromatics.

The catalytic aromatization unit includes an aromatization catalyst. The aromatization catalyst may be a zeolitic catalyst or a zeolite-supported catalyst. Non-limiting examples of zeolite-supported catalysts include Ga-ZSM-5, Zn-ZSM-5, Pt-ZSM-5, Mo-ZSM-5, Ru-ZSM-5, and Re-ZSM-5. ZSM-5 zeolites are a type of pentasil zeolite which is a shape-selective catalyst exhibiting specific adsorption and diffusion characteristics, and generally has high thermal stability and has hydrophobicity because it has high ratio of SiO2/Al₂O₃.

The following includes definitions of various terms and phrases used throughout this specification.

The terms “catalyst” and “aromatization catalyst” are used interchangeably herein. An “aliphatic group” is an acyclic or cyclic, saturated or unsaturated carbon group, excluding aromatic compounds. An aliphatic group can include 1 to 50, 2 to 25, or 3 to 10 carbon atoms. A linear aliphatic group does not include tertiary or quaternary carbons. A branched aliphatic group includes at least one tertiary and/or quaternary carbon. A cyclic aliphatic group includes at least one ring in its structure. The phrase “essentially unreacted” means that at least 90% of a substance remains chemically intact. For example, when shale gas comprising methane is reacted and the methane remains essentially unreacted, at least 90% the methane component in the shale gas remains chemically intact during the reaction(s), whereas other components may chemically react during the reaction(s).

An “alkyl group” is a linear or branched, substituted or unsubstituted, saturated hydrocarbon. In the context of the present invention, an alkyl group has 1 to 50, 2 to 30, 3 to 25, or 4 to 20 carbon atoms. Alkyl groups in the context of the present invention include all isomers and all substitution types unless otherwise stated. For example, butyl includes n-butyl, isobutyl, and tert-butyl; pentyl includes n-pentyl, 1-methylbutyl, 2-methylbutyl, 3-methylbutyl, 1-ethylpropyl, and neopentyl. Non-limiting examples of alkyl group substituents include halogen, hydroxyl, alkyoxy, haloalkyl, haloalkoxy, carboxylic acid, ester, amine, amide, nitrile, acyl, thiol, and thioether.

An “aryl group” or an “aromatic group” is a substituted or unsubstituted, mono- or polycyclic hydrocarbon with alternating single and double bonds within each ring structure. Non-limiting examples of aryl group substituents include alkyl, halogen, hydroxyl, alkyoxy, haloalkyl, haloalkoxy, carboxylic acid, ester, amine, amide, nitrile, acyl, thiol and thioether.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “vol. %,” or “mol. %” refers to a weight, volume, or molar percentage of a component, respectively, based on the total weight, the total volume, or total moles of a material, that includes the component. In a non-limiting example, 10 grams of component in 100 grams of the material is 10 wt. % of component.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

The term “substantially” and its variations are defined to include ranges within 10%, within 5%, within 1%, or within 0.5%.

The terms “inhibiting,” “reducing,” “preventing,” “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phase “consisting essentially of,” in one non-limiting aspect, basic and novel characteristics of the methods of the present invention are their abilities to efficiently produce BTX aromatic hydrocarbons from shale gas.

### BRIEF DESCRIPTION OF THE FIGURES

FIG. 1 is a schematic of the inventive process. Water and hydrates are removed from shale gas, and the crude shale gas, having methane as its primary component, is provided to an aromatics reactor. At least a portion of the ethane is converted into aromatic hydrocarbons. The aromatic hydrocarbons are separated from the methane, hydrogen, and unconverted C2+ components in an absorption column. The solvent is stripped from the aromatic hydrocarbons and recycled to the absorption column.

### DETAILED DESCRIPTION OF THE INVENTION

The present invention provides a process for producing valuable aromatic hydrocarbons from a crude or semi-crude shale gas stream. Although methane is the primary component of most shale gas, the fractions of other constituents, e.g., ethane, propane, nitrogen, water, and CO₂ varies based upon the gas deposit's geographical location.

In order to convert shale gas non-methane hydrocarbons into more valuable aromatic compounds, methane is first separated from C2+ hydrocarbons using expensive processes like cryogenic distillation separation and molecular sieves filtering. According to the instant disclosure, the methane/C2+ separation can be avoided, and a crude or semi-crude shale gas stream can be used as a feed stream for a reforming process like catalytic aromatization. At least a portion of the ethane component in the shale gas stream is converted into aromatic hydrocarbons. Unreacted methane, other hydrocarbons, and hydrogen may then be easily separated from the aromatic hydrocarbons. Because methane is not separated from the shale gas stream prior to reforming, the expensive and resource-consuming shale gas C1/C2+ separation step is avoided. The present invention therefore provides a method for the production of aromatic hydrocarbons at a reduced cost.

These and other non-limiting aspects of the present invention are discussed in further detail herein.

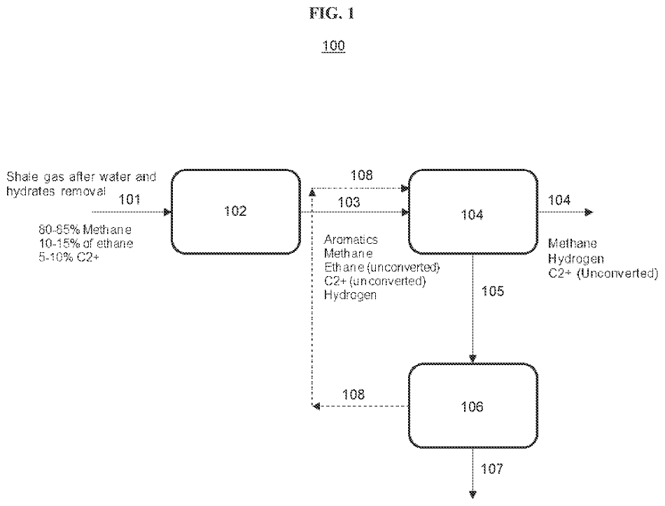

In the exemplary process 100 depicted in FIG. 1, water and hydrates are first removed from shale gas to produce a crude or semi-crude shale gas stream 101. The shale gas stream has a typical composition of 80-85 wt. % methane, 10-15 wt. % ethane and 5-10 wt. % C2+ hydrocarbons. The shale gas stream 101 is heated up to a temperature of 600-700° C. and is sent to an aromatics reactor 102 which reforms at least a portion of the ethane and higher hydrocarbons into aromatic compounds. The effluent 103 coming out the aromatics reactor 102 is cooled to a temperature of 50° C. and sent to a solvent absorption column 104 to absorb the aromatics. Effluent 103 will contain the aromatics, unconverted methane, unconverted ethane, unconverted C2+ hydrocarbons and hydrogen. The aromatic+ solvent stream 105 from the absorber is sent to series of distillation columns 106 where in BTX 107 is separated from the solvent 108, and the solvent 108 is recycled back to the absorption column 104. The BTX separation can be carried out in a Divided Wall Column in order to improve process energy efficiency. The gas stream 109 coming out of the absorption column consists primarily of methane, unconverted ethane and hydrogen. The presence of hydrogen in methane increases lower heating value (LHV) of the gas.

In the context of the present invention, embodiments 1-19 are described. Embodiment 1 is a process for the production of BTX aromatic hydrocarbons from shale gas, including reacting shale gas comprising methane, ethane, and C2+ hydrocarbons under conditions sufficient to produce a first stream comprising BTX aromatic hydrocarbons, methane, hydrogen, and unreacted ethane and C2+ hydrocarbons; contacting the first stream with a solvent to produce a second stream and a third stream, wherein the second stream comprises methane, hydrogen, and unreacted ethane and C2+ hydrocarbons, and the third stream comprises the solvent and the BTX aromatic hydrocarbons; and removing the solvent from the third stream. Embodiment 2 is the process of embodiment 1, wherein the methane in the shale gas remains essentially unreacted. Embodiment 3 is the process of either of embodiments 1 or 2, wherein water and hydrates are removed from the shale gas prior to the first step of embodiment 1 above. Embodiment 4 is the process of any of embodiments 1 to 3, wherein the step of reacting the shale gas comprises heating the shale gas to a temperature ranging from about 400° C. to about 700° C. Embodiment 5 is the process of any of embodiments 1 to 4, wherein the step of contacting the first stream with a solvent comprises contacting the first stream with an aromatic selective solvent. Embodiment 6 is the process of any of embodiments 1 to 5, wherein the aromatic selective solvent is selected from the group consisting of 1-methylnaphthalene, mono-ethylene glycol, di-ethylene glycol, tri-ethylene glycol, tetra-ethylene glycol, tetrahydrothiophene dioxide, N-methylpyrrolidone, dimethylsulfoxide, propylene carbonate, phenol, cresol, N-formylmorpholine, monomethylformamide, N-methyl-ε-caprolactam, water, and combinations thereof.

Embodiment 7 is a process for the production of BTX aromatic hydrocarbons from shale gas, including reacting shale gas containing methane, ethane, and C2+ hydrocarbons under conditions sufficient to produce a first stream containing BTX aromatic hydrocarbons, methane, hydrogen, and unreacted ethane and C2+ hydrocarbons, then contacting the first stream with a solvent to produce a second stream and a third stream, wherein the second stream contains methane, hydrogen, and unreacted ethane and C2+ hydrocarbons, and the third stream contains the solvent and the BTX aromatic hydrocarbons, distilling the third stream to produce a product stream comprising the BTX aromatic hydrocarbons and a fourth stream comprising the solvent; and recycling the fourth stream to the step of contacting the first stream with a solvent. Embodiment 8 is the process of embodiment 7, wherein the methane in the shale gas remains essentially unreacted. Embodiment 9 is the process of either of embodiments 7 or 8, wherein water and hydrates are removed from the shale gas prior to the first step of embodiment 7 above. Embodiment 10 is the process of any of embodiments 7 to 9, wherein the step of reacting the shale gas includes heating the shale gas to a temperature ranging from about 400° C. to about 700° C. Embodiment 11 is the process of any of embodiments 7 to 10, wherein the step of contacting the first stream with a solvent includes contacting the first stream with an aromatic selective solvent. Embodiment 12 is the process of embodiment 10, wherein the aromatic selective solvent is selected from the group consisting of 1-methylnaphthalene, mono-ethylene glycol, di-ethylene glycol, tri-ethylene glycol, tetra-ethylene glycol, tetrahydrothiophene dioxide, N-methylpyrrolidone, dimethylsulfoxide, propylene carbonate, phenol, cresol, N-formylmorpholine, monomethylformamide, N-methyl-ε-caprolactam, water, and combinations thereof. Embodiment 13 is the process of embodiment 12, further comprising the step of distilling the BTX aromatic hydrocarbons to recover benzene.

Embodiment 14 is a process for the production of BTX aromatic hydrocarbons from shale gas, including reacting shale gas containing methane, ethane, and C2+ hydrocarbons under conditions sufficient to produce a first stream containing BTX aromatic hydrocarbons, methane, hydrogen, and unreacted ethane and C2+ hydrocarbons, then separating the first stream to produce a second stream and a third stream, wherein the second stream comprises methane and hydrogen, the third stream comprises the BTX aromatic hydrocarbons; wherein the methane in the shale gas remains essentially unreacted. Embodiment 15 is the process of embodiment 14, wherein water and hydrates are removed from the shale gas prior to the first step of embodiment 14 above. Embodiment 16 is the process of either of embodiments 14 or 15, wherein the step of reacting the shale gas occurs in a catalytic aromatization unit. Embodiment 17 is the process of embodiment 16, wherein the catalytic aromatization unit comprises a catalyst selected from the group consisting of Ga-ZSM-5, Zn-ZSM-5, Pt-ZSM-5, Mo-ZSM-5, Ru-ZSM-5, and Re-ZSM-5. Embodiment 18 is the process of any of embodiments 14 to 17, and includes heating the shale gas to a temperature ranging from about 400° C. to about 700° C. Embodiment 19 is the process of any of embodiments 14 to 18, wherein the step of separating the first stream comprises cooling the first stream to a temperature ranging from about 30° C. to about 100° C.

### Simulated Example

Water and hydrates will be removed from shale gas to provide a shale gas stream containing methane and ethane which will be sent through an aromatization reactor at a pressure of 5-10 bara a rate of 80.0 moles/hr methane and 20.0 moles/hr ethane, with no propane or butane content. The shale gas stream will be heated up to a temperature in the range of 600-700° C. prior to entering the aromatization reactor to reform at least a portion of the ethane and higher hydrocarbons into aromatic compounds. The effluent from the aromatization reactor has the following composition shown in Table 1:

TABLE 1

| | | |
|---|---|---|
| Methane | 86.0 | moles/hr |
| Ethane | 5.0 | moles/hr |
| Propane | — | moles/hr |
| Butane | — | moles/hr |
| Ethylene | 0.9 | moles/hr |
| Benzene | 2.0 | moles/hr |
| Toluene | 0.6 | moles/hr |
| Xylene | 0.2 | moles/hr |
| Naphthalene | 0.2 | moles/hr |
| Hydrogen | 21.0 | moles/hr |
| Coke | 3.0 | moles/hr |
| Total | 115.8 | moles/hr |

The effluent from the aromatization reactor will then be cooled to a temperature of 50° C. and sent to an absorption column at a pressure of 5-10 bara to absorb the aromatics (BTX). A gas stream exits the absorber and has the content shown in Table 2:

TABLE 2

| | | |
|---|---|---|
| Methane | 86.0 | moles/hr |
| Ethane | 5.0 | moles/hr |
| Propane | — | moles/hr |
| Butane | — | moles/hr |
| Ethylene | 0.9 | moles/hr |
| Hydrogen | 21.0 | moles/hr |

The remaining effluent from the absorber is sent to a separator to separate the solvent, which is recycled to the absorber at a rate of approximately 300-600 kg/hr. The solvent in this example is mono-ethylene glycol, and liquid products in the additional effluent from the absorber are separated. This effluent was shown to have the composition shown in Table 3:

TABLE 3

| | | |
|---|---|---|
| Benzene | 2.0 | moles/hr |
| Toluene | 0.6 | moles/hr |
| Xylene | 0.2 | moles/hr |
| Naphthalene | 0.2 | moles/hr |

Analysis reveals that this example provides carbon selectivities as follows:

| Component | Selectivity, Carbon % |
|---|---|
| Methane | 20 |
| Ethylene | 6 |
| Benzene | 40 |
| Toluene | 15 |
| Xylene | 4 |
| Heavies | 5 |
| Coke | 10 |

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

1. A process for the production of BTX aromatic hydrocarbons from shale gas, the process comprising:
(a) reacting shale gas comprising methane, ethane, and C2+ hydrocarbons under conditions sufficient to produce a first stream comprising BTX aromatic hydrocarbons, methane, hydrogen, and unreacted ethane and C2+ hydrocarbons;
(b) contacting the first stream with a solvent to produce a second stream and a third stream, wherein the second stream comprises methane, hydrogen, and unreacted ethane and C2+ hydrocarbons, and the third stream comprises the solvent and the BTX aromatic hydrocarbons; and
(c) removing the solvent from the third stream.

2. The process of claim 1, wherein water and hydrates are removed from the shale gas prior to step (a).

3. The process of claim 2, wherein water and hydrates are removed from the shale gas prior to step (a).

4. The process of claim 2, wherein the step of reacting the shale gas comprises heating the shale gas to a temperature ranging from about 400° C. to about 700° C.

5. The process of claim 2, wherein the step of contacting the first stream with a solvent comprises contacting the first stream with an aromatic selective solvent.

6. The process of claim 5, wherein the aromatic selective solvent is selected from the group consisting of 1-methylnaphthalene, mono-ethylene glycol, di-ethylene glycol, tri-ethylene glycol, tetra-ethylene glycol, tetrahydrothiophene dioxide, N-methylpyrrolidone, dimethylsulfoxide, propylene carbonate, phenol, cresol, N-formylmorpholine, monomethylformamide, N-methyl-ε-caprolactam and water, or combinations thereof.

7. The process of claim 1 wherein the solvent is removed from the third steam by distilling the third stream to produce a product stream comprising the BTX aromatic hydrocarbons and a fourth stream comprising the solvent; and
(d) recycling the fourth stream to step (b).

8. The process of claim 7, wherein water and hydrates are removed from the shale gas prior to step (a).

9. The process of claim 8, wherein water and hydrates are removed from the shale gas prior to step (a).

10. The process of claim 7, wherein the step of reacting the shale gas comprises heating the shale gas to a temperature ranging from about 400° C. to about 700° C.

11. The process of claim 7, wherein the step of contacting the first stream with a solvent comprises contacting the first stream with an aromatic selective solvent.

12. The process of claim 11, wherein the aromatic selective solvent is selected from the group consisting of 1-methylnaphthalene, mono-ethylene glycol, di-ethylene glycol, tri-ethylene glycol, tetra-ethylene glycol, tetrahydrothiophene dioxide, N-methylpyrrolidone, dimethylsulfoxide, propylene carbonate, phenol, cresol, N-formylmorpholine, monomethylformamide, N-methyl-ε-caprolactam and water, or combinations thereof.

13. The process of claim 12, further comprising the step of distilling the BTX aromatic hydrocarbons to recover benzene.

14. The process of claim 1, wherein the step of reacting the shale gas comprises heating the shale gas to a temperature ranging from about 400° C. to about 700° C.;
wherein the step of separating the first stream comprises cooling the first stream to a temperature ranging from about 30° C. to about 100° C.; and
wherein the step or reacting the shale gas occurs in a catalytic aromatization unit.

15. The process of claim 14, wherein water and hydrates are removed from the shale gas prior to step (a).

16. The process of claim 2, wherein the step of reacting the shale gas comprises heating the shale gas to a temperature ranging from about 400° C. to about 700° C.

17. The process of claim 2, wherein the step of contacting the first stream with a solvent comprises contacting the first stream with an aromatic selective solvent.

18. The process of claim 5, wherein the aromatic selective solvent is water.

19. The process of claim 8, wherein the step of reacting the shale gas comprises heating the shale gas to a temperature of 400° C.

20. The process of claim 8, wherein the step of contacting the first stream with a solvent comprises contacting the first stream with an aromatic selective solvent, wherein the aromatic selective solvent is water.
